(19) United States
(12) Patent Application Publication
(10) Pub. No.: US 2004/0014024 A1
(43) Pub. Date: Jan. 22, 2004

(54) Screening assay for antagonists of FGFR-mediated malignant cell transformation and tumor formation

(76) Inventors: Avner Yayon, Moshav Sitria (IL); Reznitsky Dalya, Nes Ziona (IL); Ben-Levy Rachel, Doar NaEvtach (IL)

Correspondence Address: WINSTON & STRAWN, PATENT DEPARTMENT, 1400 L STREET, N.W., WASHINGTON, DC, 20005-3502, US

(21) Appl. No.: 09/921,279

(22) Filed: Aug. 2, 2001

Related U.S. Application Data
(63) Continuation of application No. PCT/IL00/00071, filed on Feb. 3, 2000.

(30) Foreign Application Priority Data
Feb. 4, 1999 (IL) 128380

(51) Int. Cl. C12Q 1/00; G01N 33/53; G01N 33/567

(52) U.S. Cl. 435/4; 435/7.2

(57) ABSTRACT

In vitro and in vivo screening assays for antagonists of fibroblast growth factor receptor (FGFR)-mediated malignant cell transformation are provided using stable cell line genetically engineered to express a recombinant wild type on constitutively active mutant FGFR selected from FGFR1, FGFR2 and FGFR3, the malignant potential of the cell lines being modulated by the FGFR.

### CROSS-REFERENCE TO RELATED APPLICATIONS

This application is a continuation of the U.S. national stage designation of International application PCT/IL00/0071 filed Feb. 3, 2000, the content of which is expressly incorporated herein by reference thereto.

### FIELD OF THE INVENTION

The present invention relates to the use of stable cell lines genetically engineered to express a recombinant fibroblast growth factor receptor (FGFR) selected from FGFR1, FGFR2 and FGFR3, wherein the malignant potential of said cell line is modulated by said FGFR, in in vitro and in vivo screening assays for antagonists of FGFR-mediated malignant cell transformation and tumor formation and progression, and to some such genetically engineered cells.

### BACKGROUND OF THE INVENTION

FGF receptors (FGFRs) are high-affinity receptors for the fibroblast growth factors. These factors have a diverse role in cell growth, differentiation and other biological processes, their precise function being dependent on the target cell and development stage. It has been found that mutations in FGFRs cause a variety of disorders. For example, FGFR1 and FGFR2 mutations occur in craniosynostoses, and mutations in FGFR3 have been implicated in skeletal dysplasias. Achondroplasia, the most common form of human dwarfism is caused by a point mutation (G380R substitution) in the transmembrane domain of the FGFR3 gene. Two further dwarfism syndromes, hypochondroplasia and thanatophoric dysplasia (types I and II) are also due to single mutations in the FGFR3 gene (Webster, M. K. and Donoghue, D. J. (1997) Trends in Genetics 13: 178-182).

In the case of achondroplasia, it has been suggested that the aforementioned mutation causes ligand-independent constitutive activation of the receptor (Webster, M. K. and Donoghue, D. J. (1996) EMBO J. 15: 520-527; Li, Y. et al. (1997) Oncogene 14: 1397-1406). Recent results, however, indicate that the pathogenic mechanism may in fact involve ligand-dependent signal transduction of the mutated receptor (Thompson, L. M. et al. (1997) Mol. Cell. Biol. 17: 4169-4177).

While some signal transduction pathways induced by FGFR1 and FGFR2 have been studied, the signaling pathways used by FGFR3 have not yet been resolved. Both ligand-dependent and ligand-independent signaling pathways activated by FGFR3 have been investigated. Using an in vitro model, it was found that following receptor stimulation, there is intracellular activation of the ERK and JNK members of the MAPK superfamily pathways as well as signaling through PLCγ and STAT1.

Contrary to the activity of the other known FGFRs, it is now believed, both from in vitro studies, and from the knowledge of disease states in which FGFR3 is implicated, that FGFR3 is responsible for mainly inhibitory effects on cell proliferation. The development of a screening system for the detection of antagonists of this clinically important receptor system is therefore of great importance. Furthermore, in view of the role of FGFR3 in osteogenesis (as witnessed by the aforementioned dwarfism syndromes), it is of great importance to study the role of this receptor, and of antagonists thereof in chondrocyte development.

A major technical problem that has been encountered in studies of FGFR3 expression in cultured cells, is that by virtue of its powerful growth-inhibitory properties, it is nearly impossible to successfully select and maintain a cell line transfected with a construct containing a functional FGFR3 gene.

A number of prior art references relate to screening assays for identifying FGF inhibitors. For example, WO 97/38708 discloses a method for evaluating the ability of compounds to bind FGF-2 and to modulate its activity by altering e.g. FGF-2/FGFR1 interaction. The method involves firstly evaluating the binding of the compound to FGF-2. Compounds that bind FGF-2 are then further investigated by testing the compound on an animal tissue or cell to assess its effect on epidermal-dermal interaction.

U.S. Pat. No. 5,763,214 relates to the C and protein of FGF 11 and to a screening assay for FGF 11 inhibitors. However, no details regarding said screening assay are disclosed in said patent.

### SUMMARY OF THE INVENTION

In one aspect, the present invention provides an in vitro screening assay for antagonists of FGFR-mediated malignant cell transformation comprising the steps:

(a) providing a stable cell line genetically engineered to express a recombinant fibroblast growth factor receptor (FGFR) selected from FGFR1, FGFR2 and FGFR3, wherein the malignant potential of said cell line is modulated by said FGFR;

(b) subjecting said cell line of (i) to treatment with the corresponding FGF ligand and a candidate antagonist; and

(c) measuring an FGFR downstream signaling event, wherein an antagonist is identified by suppressing said FGFR downstream signaling event.

Any suitable cell line whose malignant potential is modulated by a FGFR may be used to generate genetically engineered cells according to the invention such as, but not being limited to, cell lines derived from muscle tissue, particularly myoblast cell lines, and more particularly cells derived from the rat L8 myoblast cell line, and chondrocyte cell lines, particularly the rat RCJ chondrocyte cell line.

These suitable cells are genetically engineered to express a recombinant fibroblast growth factor receptor (FGFR) selected from wild type or a constitutively active mutant FGFR1, FGFR2 and FGFR3. For the transfection or infection of the parental cells, expression vectors are used containing a DNA molecule coding for either the wild type receptor or a constitutively active mutant receptor. Examples of known mutations of the FGF receptors that can be used according to the invention include, but are not limited to, the following known mutations: P252R FGFR1, S252W FGFR2, S267P FGFR2, W290G FGFR2, G380R FGFR3, S371C FGFR3, G370C FGFR3, R248C FGFR3, S241C FGFR3, Y373C FGFR3, and K650E FGFR3. When the FGFR is the FGFR3, the mutation is preferably the human achondroplasia G380R substitution.

In one embodiment, the FGFR is expressed in the genetically engineered cells under the control of a non-regulatable promoter. In another embodiment, the FGFR is expressed under the control of a regulatable promoter such as, but not being limited to, a tetracycline-responsive promoter, preferably a tetracycline-repressible promoter.

In one preferred embodiment, there are provided rat L8 myoblast cell lines genetically engineered by transfection or infection with an expression vector containing a encoding the wild type FGFR1, FGFR2 or FGFR3 or a constitutively active mutant FGFR1, FGFR2 or FGFR3. Preferably, the FGFR is FGFR3 either wild type or the achondroplasia mutant comprising the G380R substitution.

In another preferred embodiment, there are provided chondrocyte RCJ cell lines genetically engineered by transfection or infection with an expression vector containing a DNA encoding the wild type FGFR1, FGFR2, or FGFR3 or a constitutively active mutant FGFR3, preferably the mutant FGFR3 comprising the G380R substitution.

In the in vitro screening assay of the invention, the cells are treated with the corresponding FGF ligand of the FGFR. For example, the ligand of FGFR3 is FGF9. The interaction of the FGFR with its ligand initiates a FGFR downstream signaling event that can be suppressed/inhibited by a candidate antagonist.

The FGFR downstream signaling event that may be measured in the in vitro screening assay may be: (i) the FGFR tyrosine phosphorylation; (ii) activation of one or more intracellular proteins involved in signal transduction pathways of receptor tyrosine kinases selected from STAT1, JNK, PLCγ, ERK, STAT5, P13K, PKC, FRS2 and/or GRB2; and/or (iii) a cell differentiation-related effect.

In one preferred embodiment, the downstream signaling event is activation of jun kinase (INK), that is suppressed by the candidate antagonist.

In another preferred embodiment, the downstream signaling effect is a cell differentiation-related effect preferably selected from cell aggregation, the formation of nodules, the formation of cartilage, or two or more of said effects (Linstrum, G. P. et al. J. Histochem. Cytochem. 1999, 47: 1-6), such effects being detectable by light microscopy, turbidimetry, or flow cytometry.

In yet a further embodiment of the in vitro screening assay of the invention, the cell-differentiation effect measured is a change in the expression at RNA or protein levels of bone sialoprotein (1998 J Bone Miner. Res., 13(12): 1852-61), of matrilin-3 (1998 Genomics 53(3):391-4), of type X collagen (1998 Cell Tissue Res 293(2):357-64), the murine 4-1BB or the human ILA gene (1997 Osteoarthritis Cartilage 5(6):394-406), type II collagen and/or MGP (1997 J Bone Miner Res 12(11):1815-23), and the like.

It has further been found, according to the present invention, that cells genetically engineered to express a recombinant FGFR selected from FGFR1, FGFR2 and FGFR3, may have a different tumorigenic potential compared to the parental cells. Thus, non-tumorigenic parental cells such as the rat L8 myoblast cell line become tumorigenic when genetically engineered to express a recombinant FGFR, and tumorigenic parental cells such as the rat chondrocyte RCJ cell line become non-tumorigenic when genetically engineered to express a recombinant FGFR. This change in tumorigenic phenotype of the cells can be used to establish an in vivo screening assay for antagonists of FGFR-mediated malignant cell transformation and tumor formation and progression.

Thus, in another aspect, the present invention relates to an in vivo screening assay for antagonists of FGFR-mediated malignant cell transformation and tumor formation and progression, said assay comprising the steps:

(a) providing a stable cell line genetically engineered to express a recombinant wild type or constitutively active mutant fibroblast growth factor receptor (FGFR) selected from FGFR1, FGFR2 and FGFR3, wherein the malignant potential of said cell line is modulated by said FGFR;

(b) implanting or injecting said cells of (i) expressing said recombinant FGFR into a non-human animal;

(c) administering a candidate antagonist to said animal, either concomitantly with said cells of step (ii) or thereafter; and

(d) evaluating the formation of tumors in said animal, wherein an antagonist is identified as a suppressor of FGFR-induced tumor formation and progression or as an enhancer of FGFR-suppressed tumor formation and progression.

The non-human animal is preferably a mammal, preferably a rodent. The immune system of the non-human animal is preferably deficient in one or more aspects. More preferably, the animal is a mouse, most preferably, a SCID or nude mouse. The candidate antagonist is administered together with the genetically engineered cells or up to 21 days thereafter and the evaluation of the tumor size is carried out generally 1 to 6 weeks after implantation/injection of the genetically engineered cells into the mice.

In one preferred embodiment, the in vivo screening assay of the invention is carried out with tumorigenic genetically engineered rat L8 myoblast cells expressing a constitutively active mutant FGFR1, FGFR2 or FGFR3, most preferably the mutant FGFR3 comprising the G380R substitution, that are implanted or injected into nude mice, and a decrease in tumor formation and progression is observed in animals when an inhibitor of FGFR3 is administered to the animal.

In another embodiment, the in vivo screening assay of the invention is carried out in nude mice bearing non-tumorigenic genetically engineered rat RCJ chondrocyte cells expressing a wild type or constitutively active mutant FGFR1, FGFR2 or FGFR3, and an increase in tumor formation and progression is observed when an inhibitor of said FGFR is administered to the animal.

In still another aspect, the invention provides a stable cell line whose malignant phenotype is modulated by a FGFR selected from FGFR1, FGFR2 and FGFR3, said cell line being selected from genetically engineered rat myoblast L8 cells and rat chondrocyte RCJ cells expressing a recombinant wild type or constitutively active mutant FGFR1, FGFR2 and FGFR3 under a regulatable or a non-regulatable promoter, and progenies thereof.

In one embodiment, the stable cell lines are genetically engineered rat myoblast L8 cells expressing the wild type FGFR3 and the G380R mutant FGFR3, herein designated L8-hWTR3-34 and L8-hAchR3, respectively, and deposited at the Collection Nationale de Cultures de Microorganismes (CNCM), Institute Pasteur, Paris, France, on Feb. 01, 2000, under Accession Nos. I-2381 and I-2382, respectively, and progenies thereof

In another embodiment, the stable cell lines are genetically engineered rat chondrocyte RCJ cells expressing the G380R mutant FGFR3, wild type FGFR1, wild type FGFR2, and wild type FGFR3, herein designated RCJ-13 M14, RCJ-13 R1-1, RCJ-13 R2-2, and RCJ-13 W11, respectively, deposited at the CNCM on Feb. 2, 1999, under Accession Nos. I-2122, I-2123, I-2124, and I-2125, respectively, and progenies thereof.

All the above and other characteristics and advantages of the invention will be further understood from the following illustrative and non-limitative examples of preferred embodiments thereof.

### BRIEF DESCRIPTION OF THE DRAWINGS

FIG. 1 shows screening of RCJ clones expressing the tetracycline tet-off transactivator by transient transfection of tet-beta-gal reporter construct (designated tTA-9, tTA-13, tTA-14, tTA-15), in the presence (+) and in the absence (−) of tetracycline.

FIG. 2 shows the expression levels of FGFR3, analyzed by Western blotting with polyclonal antibodies to FGFR3, in four stable RCJ clones expressing wild type (W11, W5) or the G380R mutant (M15, M14) FGFR3, in the presence (+) and in the absence (−) of tetracycline (TET) and/or FGF9. C2: parental RCJ line clone transfected with an empty vector (negative control).

FIGS. 3A-3D show analysis by immunoblotting (IB) of signaling pathways mediated by FGFR3, performed by removal of tetracycline, stimulating the RCJ clones of FIG. 2 with FGF9 (+) or leaving cells unstimulated (−), and probing by Western blotting with antibodies (α) directed to activated phosphorylated (P) forms of ERK (3A), JNK (3B), STAT1 (3C) and p38 SAPK (3D, P38), a kinase not induced by FGFR3. C2: negative control.

FIGS. 4A-4D show analysis by immunoprecipitation (IP) and immunoblotting (IB) of signaling pathways mediated by FGFR3. The RCJ clones of FIG. 2 were stimulated with FGF9 (+) or were unstimulated (−). FIG. 4A shows the level of FGFR3 expression in the different clones by IB with polyclonal antibodies to FGFR3 (αFGFR3). FIG. 4B shows analysis of the tyrosine phosphorylated levels of FGFR3, performed by IP of FGFR3 with αFGFR3 and IB with anti-phosphotyrosine antibodies (αP-Tyr). FIG. 4C shows analysis of association of PLCγ with FGFR3 by IP of the receptor with αFGFR3 and IB with anti-PLCγ antibodies (αPLCγ The level of PLCγ phosphorylation was analyzed by IP with α PLCγ and IB with αP-Tyr (FIG. 4D). C2: negative control.

FIG. 5 illustrates the screening of FGFR3 inhibitory compounds, in which cells of the RCJ M14 clone were treated with compounds 1 to 9 (from a collection of tyrosine kinase inhibitors) and FGF9, and inhibition of FGFR3 tyrosine phosphorylation (pTYR-R3, upper panel) and of JNK activation (pJNK, lower panel) were analyzed.

FIG. 6 shows dose-dependent inhibition of FGFR3 tyrosine phosphorylation (pTYR-R3, upper row) and of JNK activation (pJNK, lower row) using 0.25-2 μM of the inhibitory compounds 2, 3 and 5 selected from FIG. 5.

FIGS. 7A-7B show analysis by IP and IB of signaling pathways mediated by FGFR1 (7A) and the level of expression of FGFR2 in one of the stable RCJ clones (7B). FIG. 7A depicts the measurement of ERK and JNK induced by FGF9 in stable RCJ clones expressing wild type FGFR1 (R1-2 and R1-1). The clones were analyzed for expression of FGFR1 by Western Blots with antibodies to FGFR1 in the presence (+) and in the absence of (−) of tetracycline, followed by removal of tetracycline and stimulation of the cells with FGF9 and probing with antibodies directed to the activated forms of ERK and JNK (7A). FIG. 7B shows FGFR2 expression by RCJ cells expressing wild type FGFR2.

FIGS. 8A-8B show tumor formation in nude mice by L8 cells, wherein the ordinate shows tumor surface area in mm² and the abscissa indicates the time in days that elapsed after injection of the L8 cells. FIG. 8A shows tumor formation by L8 cell pools infected with LSXN virus carrying the wild type FGFR3 (L8-wt-LSXN) or the G380R mutant FGFR3 (L8-Ach-LSXN). L8 (control). L8:LSXN—cells infected with empty LSXN virus. FIG. 8B depicts tumor formation by stable L8 clones transfected with pcDNA3 expressing wild type FGFR3 (clones L8-wt.34, L8-wt.16, L8-wt.15) or the G380R mutant FGFR3 (clones L8-Ach.31, L8-Ach.3, L8-Ach.14-1). L8 (control).

FIGS. 9A-9C show tumor formation in nude mice by RCJ cells expressing wild type FGFR3 (W) or the G380R mutant FGFR3 (M). C2: parental RCJ cells (control). 9A: clones C2, M15, W11; 9B: clones tTA13, W2, M16; 9C: C2 and W11 with and without doxycycline.

FIGS. 10A-10D depict the effect of FGFR3 induction on RCJ cell aggregation.

### DETAILED DESCRIPTION OF THE INVENTION

For purposes of clarity and as an aid in the understanding of the invention, as disclosed and claimed herein, the following terms and abbreviations are defined:

wt: wild-type; FGF: fibroblast growth factor; FGFR: FGF receptor; FGFR3: FGF receptor 3; IB: immunoblotting; IP: immunoprecipitation

Constitutive promoter or non-regulatable promoter: A promoter for expression of FGFR, e.g. FGFR3, that is non-repressible. The promoter may be optionally a strong promoter, such as the CMV promoter, MPSV promoter or the human elongation factor ELF-1 promoter. The promoter may also be inducible, for instance, by TPA treatment.

Regulatable promoter: A promoter that is conveniently regulatable by an exogenous agent that may be added to the cell expressing the FGFR, e.g. FGFR3, under the control of said regulatable promoter. The promoter is preferably a repressible promoter, such as the tetracycline responsive promoter described by Gossen et al., PNAS 1992, described further in Gossen, Trends Biochem Sci. Dec. 18, 1993(12):471-5, and references therein, and yet further described in U.S. Pat. Nos. 5,650,298 and 5,589,362, 5,814,618, 5,807,731, and 5,789,156, all to Bujard et al., all these publications being herein incorporated by reference in their entirety. It may also be an inducible promoter having low background activity. Examples of suitable promoters include the metallothionein promoter (e.g., Nephrol Dial Transplant. 13:1420, 1998), and the CYP1A1 promoter (J Cell Sci 109:2619, 1996, Proc Natl Acad Sci U S A. 92:11926, 1995).

FGFR antagonist: Molecules that specifically interact and decrease FGFR signal transduction. These molecules may interact with the FGF receptor directly, with the FGF ligand (e.g. FGF9), or with both, and influence the binding or receptor aggregation characteristics of the growth factor to the receptor. The molecule may also interact with the receptor at the intracellular domain thereof, or with downstream signaling factors.

The FGFR pathway: Comprises all events from the binding of FGF ligand to the FGFR receptor to the final effect thereof. This includes receptor-ligand interaction, receptor crosslinking, receptor modulation, receptor modification (e.g., phosphorylation), intracellular receptor-protein interactions, and interactions of further downstream signaling components with each other and with other cellular components.

Thus, in one aspect, the present invention relates to screening assays for FGFR1, FGFR2 or FGFR3 antagonists using cell lines stably expressing wild type or mutant FGFR1, FGFR2 or FGFR3. The technical problem of making stable cell lines expressing such FGFR is surprisingly overcome by the finding that cells wherein FGFR signals for inhibition of cell growth may be stably transfected or infected with an expression vector containing molecules coding for FGFR and a regulatable promoter system. When maintaining the cell line, the FGFR remains downregulated by means of the regulatable promoter that drives expression of said FGFR. When performing an experiment, e.g., screening for FGFR antagonists according to the invention, the receptor is expressed by up-regulating the regulatable promoter. It has further been surprisingly found that the growth inhibitory effect of FGFR in these cells does not interfere with the function of FGFR in these cells as required for the screening assay of the invention.

The stable FGFR expressing cell lines are used in an in-vitro screening assay of the invention, whereby a cell stably expressing FGFR under the control of a regulatable promoter is cultured, the regulatable promoter is up-regulated to allow for sufficient FGFR expression, and ligand is added. After a certain time period, FGFR signaling effects are measured. When a compound is added to the assay, either before, together with, or after addition of the ligand, the inhibitory effect of said compound on the FGFR signaling effect can be easily determined, compared to the control reaction where only ligand is added. According to the invention, a second control reaction may be performed wherein the regulatable promoter is not upregulated. This reaction allows the determination whether the effect of the compound is due specifically to interaction with the FGFR signaling pathway.

As stated, the invention also provides an in vivo screening assay for FGFR antagonists. It has been surprisingly found that when cell lines stably transfected with FGFR are injected into animals, their tumorigenic potential depends upon the extent of FGFR expression. In the in vivo screening method of the invention, a parental cell line is injected into an animal, and the tumorigenic potential thereof is determined. The stable FGFR expressing cell line is injected in a second animal or group of animals, and the tumorigenic potential thereof is measured. In a third animal or group of animals, a compound is administered to the animal, either before, at the same time, or after injection of the FGFR-expressing cell line. A change in the tumorigenic potential of the FGFR expressing cell line caused by the compound is thus due to interaction of the compound with the FGFR signal transduction pathway or ligand-receptor interaction. As a specificity control, the regulatable promoter that drives expression of the FGFR in the cell line may be downregulated in the animal. A compound that specifically interacts in the FGFR pathway will have no effect when introduced into an animal where the regulatable promoter driving FGFR expression is downregulated. The FGFR is preferably FGFR3.

Stable cell lines expressing recombinant FGFR, e.g. FGFR3, for use in the above in vivo screening assay do not necessarily have to express FGFR under the control of a regulatable promoter. The inventors have surprisingly found that by selecting a cell line that responds to recombinant expression of FGFR therein by enhanced cell growth, a stable FGFR expressing cell line can be established wherein FGFR expression is driven by a nonregulatable promoter.

According to the invention, the cell lines may be derived from cell lines of mesenchymal origin. Established cell lines or primary cell cultures may be used. Such cell lines may be derived from a variety of different cell types, including, but not limited to cells from muscle tissue such as myoblasts, including rat L8 myoblast cells; endothelial cells, including BAEC (bovine aortic endothelial cells); osteoblasts, including ROS (rat osteosarcoma) cells; and chondrocytes, including rat chondrocyte RCJ cells.

The FGFR cDNA may be introduced into these cells by transfection, retroviral gene transfer, viral infection, or homologous recombination. These techniques are described in detail in a large number of articles and textbooks, e.g., in the above Ausubel et al., Current Protocols in Molecular Biology, chapter 9, and Methods in Enzymology: Gene Expression Technology, Goeddel, D. V. and Gold, L. (Eds), Vol. 185, 1991, Academic Press.

In certain cells the expression of FGFR under control of a constitutive, optionally strong promoter is possible. According to the teaching of the invention, a simple test will determine whether a cell line is suitable for expression of the FGFR under the control of a non-repressible promoter. For instance, the L8 cell line may be transfected with an expression vector containing the cDNA coding for FGFR under the control of a non-inducible promoter.

Promoters suitable for expression of FGFR in cells include the cytomegalovirus promoter, for instance, as present in the pcDNA3 vector as available from Invitrogen Inc., the MPSV promoter, for instance as available in the pMPSVEH vector (Artelt et al., Gene. Sep. 7, 1988;68(2):213-9), the SV40 promoter, viral long terminal repeat-derived promoters, promoters of translation elongation factors (e.g., as described in J Biol Chem Apr. 5, 1989;264(10):5791-8), or promoters of growth factor receptors. Further information regarding possible promoters may be found e.g., in the above Methods in Enzymology: Gene Expression Technology textbook.

In certain cells expression of the FGFR3 is difficult due to its growth inhibition properties. In accordance with the teaching of the invention, FGFR3 may be introduced into these cells and expressed in stable cell lines under the control of a regulatable promoter.

Regulatable promoters that may be used include the metallothionein-1 promoter and the CYP1A1 promoter. Preferably, a tetracycline-responsive promoter is used.

The tetracycline-regulatable promoter consists of a promoter that contains the tet operator sequence, preferably a multiple copy thereof. The operator sequence may comprise one or more mutations, as described by Gossen et al. In U.S. Pat. No. 5,589,362. The use of a tetracycline-inducible promoter necessitates the expression in the cell of a transactivator, as described in the above publications by Gossen et al. The transactivator is a chimeric protein that consists of the tet repressor protein of the Tn10 transposon of E. coli, and of a eukaryotic transcriptional activator, such as the herpes virus 16 gene product. Of course, other transactivators may be used. When using a transcriptional transactivator in the tetracycline regulatable system, the promoter driving expression of the FGFR receptor must have a low background level in the absence of transactivator binding. This is achieved in the above system of Gossen et al., PNAS, 1992, by using a minimal promoter derived from the human cytomegalovirus promoter IE and containing the RNA polymerase site thereof This promoter is fused to multiple copies of the tet operator sequence. In the absence of transactivator binding, the promoter has a very low activity. In order to become regulatable, the transactivator must therefore contain a eukaryotic transcriptional activation domain.

However, it is also possible to use a strong promoter, such as the above SV40, CMV, MPSV, or the human elongation factor-1 promoter, fused to multiple copies of the tet operator sequence. The cell must then express a chimeric protein that comprises the said tet repressor domain fused to a strong eukaryotic transcriptional repressor domain. This type of system where a transcriptional repressor is regulated by tetracycline is described by Gossen et al. in U.S. Pat. No. 5,789,156. Repressors that may be used include the PRDI-BF1/Blimp-1 protein (Genes Dev 13:125, 1999) and the cut/CDP protein (Blood 93:519, 1999).

The stable cell line of the invention expressing FGFR, e.g. FGFR3, under the control of a regulatable promoter is suitable for use in a specific screening assay for FGFR antagonists according to the invention. When adherent cells are used, the confluence of the culture before commencement of the assay should be from about 60% to about 80%, and most preferably about 80%.

Said assay requires culturing said stable cell line under conditions that upregulate or downregulate the regulatable promoter. When using the tetracycline regulatable promoter described above, this is done simply by either removing tetracycline or adding same, depending upon the exact kind of promoter system used. Preferably, the cells contain the tetracycline-dependent transactivator, and the tetracycline is removed, so that the transactivator can bind and activate the regulatable promoter. When other promoters are used, e.g., the metallothionein promoter, zinc or glucocorticoids, e.g., dexamethasone, are added. After a first time period, a compound is added to the culture. After a second time period after said time point, ligand or an equivalent thereof is added to the culture. After a third time period after said time point has elapsed, the cells are harvested.

The first time period may range from zero to several days, preferably between zero and one hour, more preferably about zero. The second time period may be longer or shorter than the first time period. Preferably, the second time period is longer, more preferably, about one minute to about five hours longer than the first time period, and most preferably, about twenty minutes longer than the first time period.

Cells are stimulated by ligand, which may be any fibroblast growth factor molecule that is capable of interacting with FGFR. However, FGF derivatives or mutants may be used also. Such mutants are disclosed e.g., TW 264481, EP 645451, U.S. Pat. No. 5,252,718, U.S. Pat. No. 5,132,408, WO 90/02800, which publications are herein incorporated by reference in their entirety. When using FGF mutants or derivatives, it must be ascertained that they are capable of signaling for FGFR, e.g. FGFR3, events. This may be done using the assay for FGFR downstream effects as described further below. FGF mutants or derivatives, or other compounds capable of binding to the FGF receptor, may be used for receptor stimulation even if they are inactive by themselves. This is done by preparing dimers of said compounds, as described in WO 96/40772. Dimerization may either confer enhanced activity of compounds that are active in stimulating FGF receptors, or they may impart activity on compounds that bind FGF receptor but fail to stimulate it. Another agent that may be used to stimulate FGFR is an agonist anti-FGFR antibody.

The harvested cells are then subjected to analysis of the FGFR and signaling proteins therein. This may be done according to established procedures (e.g. Leevers, S. J. & Marshall, C. J. (1992) EMBO J. 11:569-574; Rausch, O. & Marshall, C. J. (1997) Mol. Cell. Biol. 17: 1170-1173). An assay for PLC gamma is described in Mol. Pharmacol. 42: 743, 1992. Further assays for signal transduction proteins are described e.g., in Current Protocols in Cell Biology, Juan S. Bonifacino et al. (eds), John Wiley & Sons, Inc., chapter 14. Preferably, the assay is carried out as described further below.

According to the invention, signaling proteins STAT1, JNK, ERK and PLCγ are preferred in the assay of the invention. However, other signaling components may be checked, using a cell line of the invention. Signaling components that are specifically activated by stimulation of FGFR3, as described hereinbelow for the above-mentioned signaling components, may then be selected and used in the assay of the invention. Such signaling components could be, for example, STAT5, PI3K, PKC, FRS2, GRB2 etc.

In one embodiment of the invention, the downstream signaling event is FGFR3 tyrosine phosphorylation. In another embodiment of the invention, the downstream signaling event is activation of STAT1, JNK, PLCγ, or ERK. In a further embodiment of the invention, the downstream signaling event is differentiation of said cell. Preferably, as a measure of differentiation, cell aggregation is measured, e.g. by light microscopy, turbidimetry, or flow cytometry. In a yet further embodiment of the in vitro screening assay of the invention, the expression of bone sialoprotein, of matrilin-3, of type X collagen, the murine 4-1BB or the human ILA gene, type II collagen and MGP mRNA, and the like, is measured.

The invention also provides an in vivo screening assay, wherein a number of cells according to the invention are injected into a number of non-human animals and the formation of tumors in said animals is evaluated. In certain animals, a compound to be screened is administered to the animal. Changes in the formation of tumors in animals where the compound has been introduced compared to control animals indicate that the compound interacts with the FGFR signaling pathway. Preferably, the immune system of said non-human animal is deficient in one or more aspects, to facilitate tumor growth. Preferred animals are mammals, e.g., rodents, more preferably mice, for example, a nude mouse.

An illustrative example of an embodiment of the invention is an in vivo screening assay in which the animal is a nude mouse, the cells are RCJ cells expressing FGFR3 under the control of said tetracycline responsive promoter, and an increase in tumor formation is observed in animals where an inhibitor of FGFR3 signal transduction is introduced into the animal.

Another illustrative example of an in vivo screening assay is one in which the animal is a nude mouse, the cells are L8 cells expressing constitutively active mutant FGFR3, and a decrease in tumor formation is observed in animals where an inhibitor of FGFR3 is introduced into the animal.

Six cell lines according to the invention were deposited under the Budapest Treaty at the CNCM, Institut Pasteur, Paris, France: four RCJ clones on Feb. 2, 1999, under Accession Nos. I-2122, I-2123, I-2124, I-2125, and two L8 clones on Feb. 01, 2000, under Accession Nos. I-2381 and I-2382.

The parental RCJ cell line is a non-transformed rat chondrocyte cell line derived from neonatal rat calvaria (Grigoriadis et al., 1990, Dev. Biol. 142, 313-318; Grigoriadis et al., 1996, Differentiation 60, 299-307).

The parental rat myoblast L8 cell line was kindly provided by Dr. David Yaffe, Weizmann Institute of Science, Rehovot, Israel (D. Yaffe and O. Saxel, 1977, Differentiation Vol. 7, pp. 159-166).

A description of the deposited cell lines is as follows:

I-2122—Cell line RCJ-13 M14 carrying an expression vector expressing FGFR3-mutant (G380R). This vector was constructed by cloning cDNA for FGFR3-mutant into a Bam H1 site of the expression vector paHygTet1.

I-2123—Cell line RCJ-13 R1-1 carrying an expression vector expressing wild type FGFR1. This vector was constructed by cloning cDNA for FGFR1 into a Bam H1 site of the expression vector paHygTet1.

I-2124—Cell line RCJ-13 R2-2 carrying an expression vector expressing wild type FGFR2. This vector was constructed by cloning cDNA for FGFR2 into a Bam H1 site of the expression vector paHygTet1.

I-2125—Cell line RCJ-13 W11 carrying an expression vector expressing wild type FGFR3. This vector was constructed by cloning cDNA for FGFR3 into a Bam H1 site of the expression vector paHygTet1.

I-2381—Cell line L8-hWTR3-34 is a rat myoblast cell line that expresses the human wild type FGFR3. It was generated by transfection of L8 cells with pcDNA3 containing a cDNA fragment of FGFR3. Cells expressing the receptor were selected by growing the transfected cells with 0.8 mg/ml of G418 (Gibco), for 3 weeks.

I-2382—Cell line hAchR3-3 is a rat myoblast cell line that expresses the human achondroplasia mutated (G380R) FGFR3. It was generated by transfection of L8 cells with pcDNA3 containing a cDNA fragment of the achondroplasia mutated FGFR3. Cells expressing the receptor were selected by growing the transfected cells with 0.8 mg/ml of G418 (Gibco), for 3 weeks.

### EXAMPLES

General Materials and Methods

In the following, general materials and methods used in the practice of the invention are described. A number of methods are not detailed herein, as they are well known to the skilled artisan. These include e.g., genetic engineering techniques, generation of antibodies, and the like. Techniques relating to molecular biology are detailed in many articles and textbooks, for instance, in Ausubel et al., Current Protocols in Molecular Biology, Greene Publications and Wiley Interscience, New York, N.Y., 1987-1995; Sambrook et al., Molecular Cloning: A Laboratory Manual, Cold Spring Harbor Laboratory, Cold Spring Harbor, N.Y., 1989. Likewise, techniques relating to immunology, e.g., the generation of antibodies, are detailed in: Current Protocols in Immunology, John E. Coligan et al. (eds.), John Wiley & Sons.

Generation of Virus-Infected Cells Expressing FGF Receptors

The cDNA of the indicated FGFR was inserted into the viral pLSXN vector using standard techniques. As packaging cell line served the 293-T cell line, which was double-transfected with the FGFR-containing pLSXN construct and the pφ 10-40 viral vector, which provides the proteins necessary for packaging. The pLSXN and φ 10-40 vectors are available commercially from Clontech Inc., USA. Infectious virus particles containing FGFR-coding DNA sequences were then harvested and used to infect L8 or RCJ cells. Pools of infected cells were used for further study as described hereinbelow.

Generation of Stable Cell Lines

The RCJ cell line is a non-transformed rat chondrocyte line, which has been subcloned from a pluripotent mesodermal stem cell line derived from neonatal rat calvaria (Grigoriadis et al., 1990, Dev. Biol 142, 313-318). RCJ clone 3.1C5.18 (Grigoriadis et al., 1996. Differentiation 60, 299-307) was kindly provided by Dr. J. Auburn. These cells were transfected with a plasmid encoding for the tetracycline tet-off trans-activator, and carrying the neomycin resistant gene (pTet-Off, Clontech Cat. No. K1620-A). G418 resistant clones were assayed for expression of the transactivator by transient transfection of tet-beta-gal construct pUHG16-3 (Resnitzky et al., 1994, MCB 14, 1669-1679) with and without tetracycline. RCJ derived clone tTA-13 showed the highest fold induction of beta-gal activity, upon tetracycline removal, and was therefore chosen for the next step.

RCJ derived tTA-13 cells were transfected with expression vectors capable of expressing wt and mutant FGFR3 in a tetracycline repressible manner. The single nucleotide exchange G380R (achondroplasia) mutation of FGFR3, was made by PCR-mediated mutagenesis, using the PCR primer 5′ CAGGAATTCTCAGCTACAGGGTGGGCTTC. The cDNA sequences coding for FGF receptors were as published (FGFR1, Genbank acc. No. M34641, FGFR2, Genbank acc. No. X52832, FGFR3, Keegan et al. (1991) Proc. Natl. Acad. Sci. U S A. 88, p. 1095-9). For cloning into expression vectors or viral vectors, FGF receptor cDNAs were truncated at about 100 nucleotides 5′ to the ATG translation start codon. Further, the 3′ noncoding region was removed completely, using PCR cloning.

The cDNA for either wt or the G380R mutation of FGFR3 contained in pBluescript (Stratagen Inc., La Jolla, USA) was excised with EcoRI and XhoI, and inserted into the pcDNA3 vector (Invitrogen Inc., USA). The cDNA was then excised from the pcDNA3 vector using Bam HI and inserted into the multilinker of pAHygTet1 (obtained from Dr. A. Himmler, Bender & Co. GmbH, Dr. Boehringer Street, Vienna, Austria). The cDNA sequences coding for FGFR1 or FGFR2 were similarly cloned into pAHygTet1. The pAHygTet1 vector was constructed by digesting pAHygCMV1 (Weyer, U. et al. (1993) Receptors and Channels 1: 193-200), (containing the hygromycin resistance gene under the control of the TK promoter) with SpeI. A filling-in reaction using the Klenow enzyme was then carried out, and the vector digested with Bam HI. The vector portion was then isolated by agarose gel electrophoresis. The pUHJD10-3 vector (Gossen, M. & Bujard, H. (1992) PNAS 89: 5547-61) was digested with XhoI, filled in with Klenow enzyme and further digested with BamHI. The 0.47 kB fragment was isolated by agarose gel electrophoresis, and ligated into the above-described vector portion of pAHygCMV1. This vector, containing two BamHI sites in the polylinker, was then digested with BamHI and religated, to yield pAHygTet1.

Screening for FGFR3-expressing clones in the absence of tetracycline yielded only very few clones. None of these was capable of achieving satisfactory levels of receptor expression. Following selection in hygromycin (50 μg/ml, Gibco BRL), clones were screened for ability to express FGFR3 in a tetracycline repressible manner. FGFR1 and FGFR2 stable cell lines were generated by the same procedure.

Some cells, e.g., L8 cells as described herein, may be transfected with a nonregulatable expression construct for FGFR3 expression. FGFR3 cDNA was inserted as described above, into the commercially available pcDNA3 vector. This construct was then used to transfect L8 cells and select resistance clones by standard procedures.

Immunoprecipitation and Western Blotting

The technique of immunoprecipitation is detailed in many articles and textbooks, e.g., in the above Current Protocols: Molecular Biology, chapter 6. The antibodies used for immunoprecipitation and immunoblot (Western) analysis are commercially obtainable from a number of sources, e.g., Santa Cruz Biotechnology Inc., Santa Cruz, Calif., or Bionostics Inc., Canada.

Immunoprecipitation of FGFR1 or FGFR3 was done according to standard procedures using antibodies #121 #123 (Santa Cruz Biotechnology Inc., Santa Cruz, Calif.), respectively. Western blotting was done according to standard procedures. The blots were probed either with anti-FGFR1 (αFGFR1) or anti-FGFR3 (αFGFR3) antibodies (Santa Cruz) or with anti-phosphotyrosine (αP-Tyr) 4G10 (UBI, Upstate Biotechnology Inc., U.S.A.) or with anti-activated ERK (αP-ERK) , JNK (αP-JNK), p38 SAPK (αP-P38) (Promega, Wis., USA) or with anti-phospho STAT1 (αP-STAT1) (NEB, New England Biolabs Inc., MA, USA). Immunoprecipitation of PLCγ was done according to the manufacturer's recommended procedure (UBI).

Screening of Inhibitory Compounds in RCJ Cells Overexpressing FGFR3

A cell-based assay for FGFR3 inhibition by selected anti-FGFR compounds, was developed. The RCJ-M14 cell line was grown in 6-well plate to 80% confluence. Tetracycline was removed and after 12-16 hours cells were starved without serum for 5-6 additional hours. Cells were then challenged with 2.5 μM of each compound for 20 minutes after which 100 ng/ml FGF9 (Peprotech) was added for 5 minutes. The cells were lysed in 0.5 ml lysis buffer and the cleared lysates were subjected to IP with 1 μg anti-FGFR3 antibodies (Santa Cruz #123) for at least 4 hours at 4° C. The immunecomplexes were then probed with 4G10 anti-P-Tyr mAb (UBI). In parallel, aliquots of the total protein lysates were immunoblotted with anti-pJNK (Promega) or with 4G10. Untreated FGF9-induced and non-induced lysates were included in each assay as references for compounds potency. The potency of each compound was evaluated by analyzing the intensity of the specific bands on the autoradiograms, giving it scores from 0 to 4 according to the relative inhibition. 0=no inhibition, 1=less than 25% inhibition, 2=25-50%, 3=50-75% and 4=75-100% inhibition.

Tumor Formation in Nude Mice

6-8-Week old nude mice (BalbC/Nu) were injected subcutaneously with equivalent number of parental RCJ or L8 cells or FGFR3 expressing cell lines and were followed for tumor formation for up to 60 days after injections. Inhibition of FGFR expression in vivo was done by adding 25 μg/ml doxycycline into the drinking water of mice which had previously received the RCJ cell clones by injection.

Cell Differentiation Assay

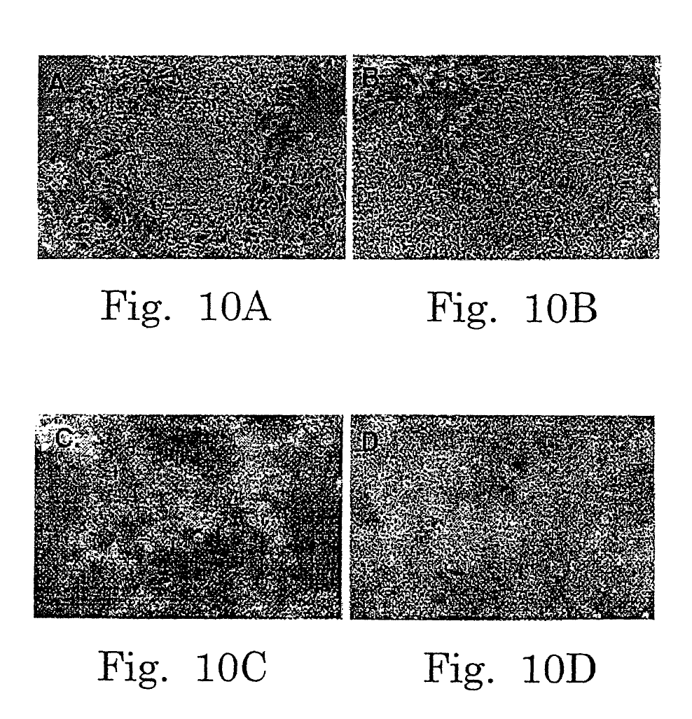

The assay was performed as previously described (Grigoriadis et al. (1996) Differentiation 60: 299-307) with slight modifications. 10,000 cells per well were seeded in 6-well plates, and incubated overnight. The medium was then replaced with medium containing additives (50 μg/ml ascorbic acid, 10 mM beta-glycerophosphate, and 0.1 μM dexamethazone), with (non-induced conditions) or without (induced conditions) tetracycline. Medium was replaced twice a week, for three weeks, after which time cartilage nodules were monitored. Induction of FGFR3 (either wild type or mutant) by tetracycline removal significantly inhibited nodules formation (FIG. 10).

### Example 1

Generation of RCJ Stable Cell Lines Expressing Human FGFR3

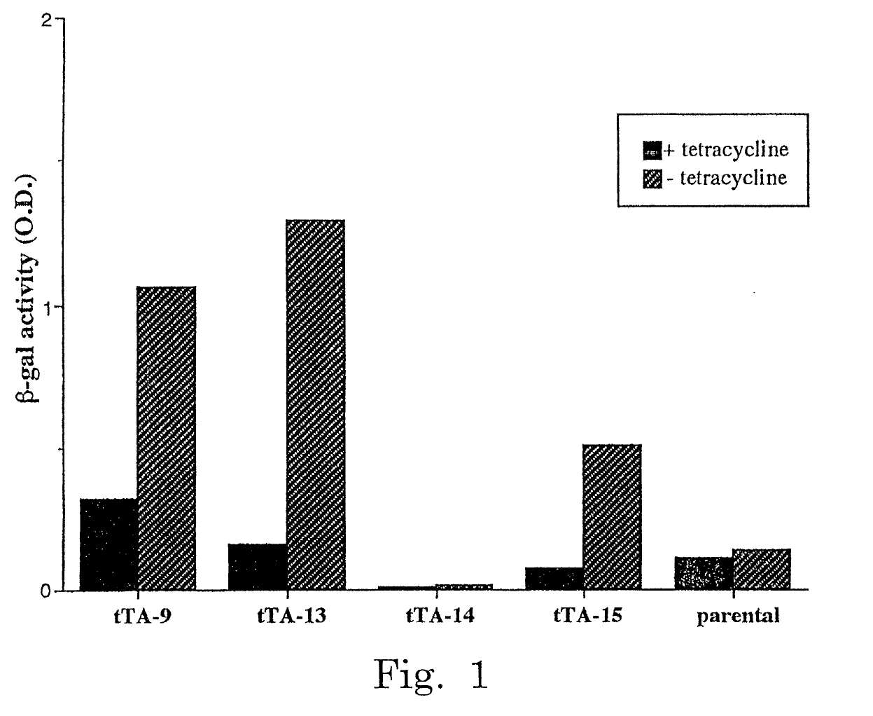

The RCJ cell line is a non-transformed rat chondrocyte line, which has been subcloned from a pluripotential mesodermal stem cell line derived from neonatal rat calvaria (Grigoriadis et al., 1990, Dev. Biol 142, 313-318). This cell line was transfected with a plasmid encoding the tetracycline tet-off transactivator and the neomycin resistance gene. G418 resistant clones were assayed for expression of the transactivator by transient transfections of tet-beta-gal reporter construct in the presence and in the absence of tetracycline. 13 clones were tested, and 3 of them were found to allow beta-gal activity in a tetracycline repressible manner. FIG. 1 shows the results of the beta-gal activity in three positive clones (tTA-9, 13, 15), and in one of the negative clones (tTA-14). Clone tTA-13 showed the highest fold induction of beta-gal activity, upon removal of tetracycline, and was therefore chosen for the next step.

The G418 resistant RCJ clones transfected with the tetracycline transactivator (indicated on the abscissa in FIG. 1) were screened for expression of the transactivator by transient transfection with the tet-beta-gal construct. Cells were assayed for beta-galactosidase activity in the presence (black bars) and absence (hatched bars) of tetracycline. The extent of beta-gal expression is given as OD values on the ordinate.

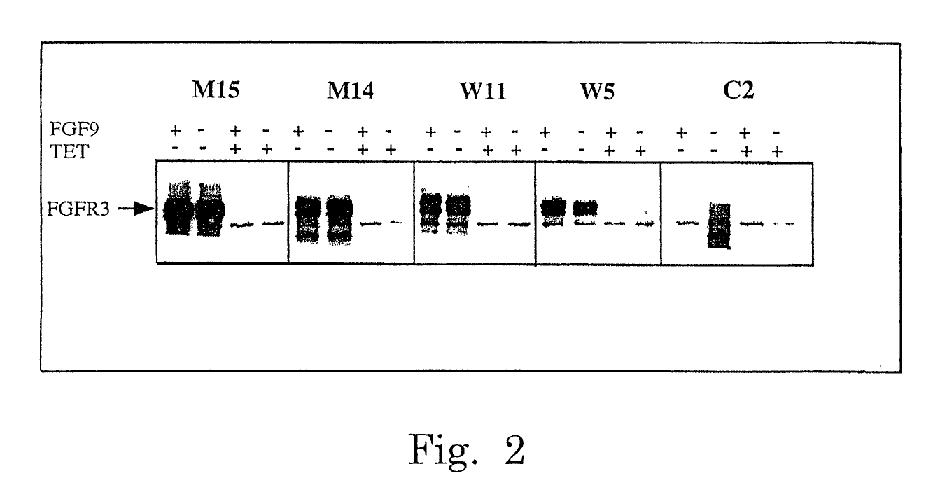

RCJ derived tTA-13 cells were transfected with expression vectors capable of expressing wild type (wt) and mutant human FGFR3 in a tetracycline repressible manner. These expression vectors were constructed by cloning the full length cDNA for either wt or the G380R mutation of FGFR3 into the Bam H1 site of the expression vector PAHygTet1, which contains the tet operator sequences upstream of a multiple cloning site, as well as the hygromycin resistance gene. Following selection in hygromycin, clones were screened for ability to express FGFR3 in a tetracycline repressible manner, by Western blot analysis, using polyclonal rabbit antibodies (Santa Cruz). About 50% of the hygromycin resistant clones isolated after this second round of transfection, were found to express FGFR3 in an inducible manner. FIG. 2 shows the expression levels of FGFR3, with and without induction, in four of . these clones, which are tightly regulated by tetracycline. This was analyzed by Western blotting with polyclonal antibodies to FGFR3. Comparable levels of receptors are expressed by clone W 11 of wild type receptor and by clone M14 of the mutant. Clone M15, transfected with mutant receptor, expresses very high levels of the mutated receptor, while clone W5, transfected with wild type receptor, expresses the lowest levels of FGFR3 among these clones. Overexpression of FGFR3 in RCJ clones in the presence and in the absence of tetracycline and/or FGF9 was analyzed by probing Western blots with FGFR3 specific polyclonal antibodies. Two of the selected wild type receptor clones, W5 and W11, are shown. Two of the selected achondroplasia G380R mutant receptor clones, M14 and M15, are also shown. C2 is a clone of the parental RCJ line transfected with an empty vector and serves as negative control.

### Example 2

Signal Transduction of FGFR3 in RCJ Cells

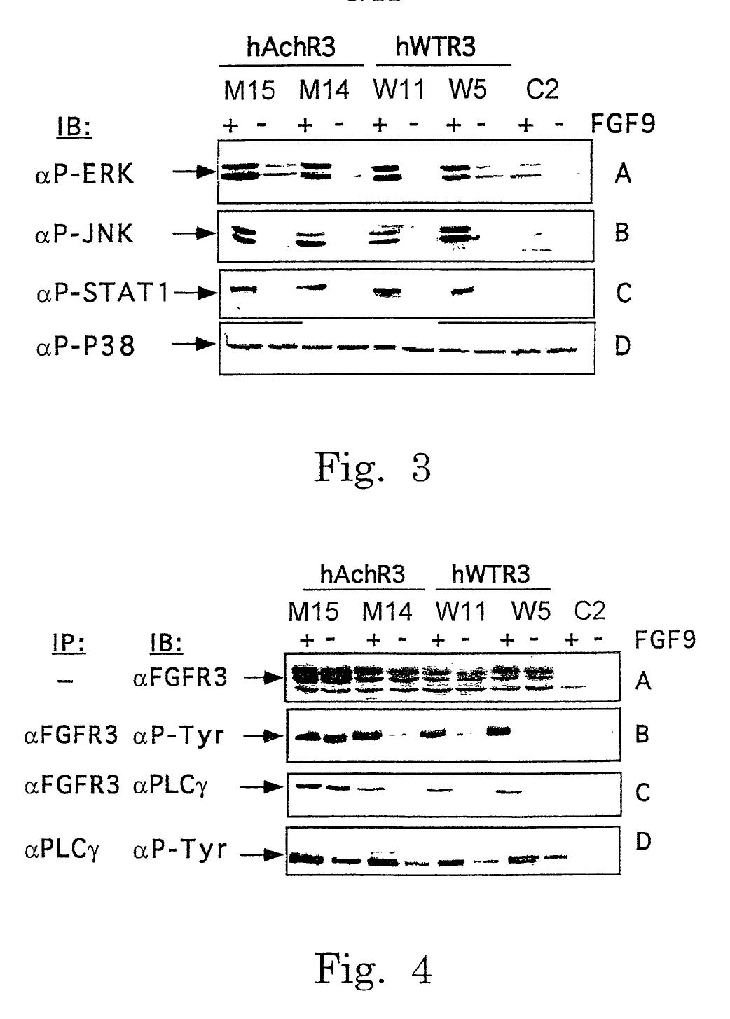

Having achieved reproducible levels of FGFR3 expression in these clones, their signal transduction properties were investigated. ERK (also known as MAPK) activity was analyzed by Western blotting with antibodies to the activated (phosphorylated) form of ERK. As shown in FIG. 3A, ERK activity is dramatically induced by FGF9 but is not exclusively mediated via exogenous FGFR3 since ligand induced activation of ERK is also observed in the C2 parental RCJ line.

Activation of stress activated ERK homologues, JNK and p38 SAPK, were also analyzed. Western blots with antibodies to the activated forms of JNK (FIG. 3B) or p38 SAPK (FIG. 3D) show that while activation of JNK is ligand dependent and mediated almost exclusively via exogenous FGFR3, p38 SAPK has some basal activity which is not induced by FGFR3 (p38 SAPK was used for comparison).

RCJ clones M14, M15, W5, W 11 and control C2 are as described in FIG. 2. Analysis of the signaling pathways mediated by FGFR3 was performed by removal of tetracycline, stimulating the cells with FGF9 (+) or leaving cells unstimulated (−), and probing by Western blotting with antibodies directed to the activated (phosphorylated) forms of ERK (FIG. 3A), JNK (FIG. 3B), STAT1(FIG. 3C), and p38 SAPK (FIG. 3D).

The above results constitute the first demonstration of activation of JNK as a signaling event downstream to FGF receptor activation. The JNK assay provides a test for FGF receptor activation with essentially zero background. In a preferred embodiment of the invention JNK is used for the evaluation of the activity of potential antagonists of FGF receptors in the screening assay of the invention.

Another signaling pathway that was analyzed is the JAK/STAT pathway. STAT1 is a transcription factor and its activation is mediated by phosphorylation on tyrosine 701. Upon phosphorylation, STAT1 dimerizes and translocates to the nucleus where it mediates its transcriptional activity. Western blots probed with specific antibodies to the activated form of phospho-STAT1 showed ligand-induced activation of STAT1 that is mediated via FGFR3 (FIG. 3C).

Analysis of the tyrosine phosphorylated levels of the receptor, performed by immunoprecipitation of FGFR3 with polyclonal antibodies and immunoblotting with anti-phosphotyrosine antibodies, revealed FGF9 dependent tyrosine phosphorylation of the receptor in all clones (FIG. 4B). FIG. 4A shows the level of FGFR3 expression in this experiment. At extremely high expression levels of the receptor as obtained in clone M15 there is a high basal tyrosine phosphorylation level which is apparently ligand independent (FIG. 4B).

Immunoprecipitation of FGFR3 also shows that PLCγ associates with tyrosine phosphorylated FGFR3 (FIG. 4C). Note that in clone M15, where constitutive tyrosine phosphorylation is observed, PLCγ is associated with the phosphorylated receptor independently of ligand induction.

In FIG. 4, RCJ clones are as described in FIG. 2. Basal (−) and FGF9 induced (+) tyrosine phosphorylation of FGFR3 was determined by immunoprecipitation of the receptor with antibodies to FGFR3 and immunoblotting with antibodies to phosphotyrosine (FIG. 4B). Association of PLCγ with FGFR3 was analyzed by immunoprecipitation of the receptor with anti-FGFR3 antibodies and immunoblotting with anti PLCγ antibodies (FIG. 4C). The level of PLCγ phosphorylation was analyzed by immunoprecipitation with anti-PLCγ antibodies and immunoblotting with anti-phosphotyrosine antibodies (FIG. 4D). The level of FGFR3 expression in the different clones, detected by anti-FGFR3 antibodies, is also shown (FIG. 4A).

Immunoprecipitation of PLCγ from all the cell lines indicated that activation of PLCγ is highly induced upon stimulation of the cells with FGF9, as shown in FIG. 4D.

### Example 3

RCJ Cells Expressing FGFR3 as a Model System to Analyze Inhibitory Compounds

Based on the results obtained we have developed a cell based assay for FGFR3 inhibition by selected anti-FGFR compounds. The RCJ-M14 cell line, which expresses high levels of the mutated FGFR3, was used for this purpose. Cells were grown in 6-well plate to 80% confluence when tetracycline was removed from the medium to allow expression of the receptor. 12-16 Hours later, cells were starved without serum for 5-6 additional hours. Cells were then challenged with the test compounds 1 to 9 (from a large collection of tyrosine kinase inhibitors) (2.5 μM) for 20 minutes after which time, FGF9 was added for 5 minutes. The cells were lysed in 0.5 ml lysis buffer and the cleared lysates were subjected to immunoprecipitation with anti-FGFR3 antibodies. The immune complexes were then probed with 4G10 anti-phosphotyrosine antibodies. In parallel, aliquots of total protein lysates were immunoblotted with anti-activated (phosphorylated) pJNK antibodies. Lysates of cells either treated or not treated with FGF9 were included in each assay as reference for compound potency. The results are shown in FIG. 5. After FGF9 stimulation, cells were harvested and subjected to immunoprecipitation with anti-FGFR3 antibodies and Western blots with anti-phosphotyrosine antibodies (upper panel). Total cell lysates were also analyzed for inhibition of JNK activation (lower panel). The numbers 1 to 9 in FIG. 5 represent different inhibitory compounds. The potency of each compound and the controls was evaluated by analyzing the intensity of the specific bands on the autoradiograms, assigning scores from 0 to 4 according to the relative inhibition, i.e., 0=no inhibition, 1=less than 25% inhibition, 2=25-50%, 3=50-75% and 4=75-100% inhibition. An example of such an analysis is shown in FIG. 5. Compounds 1, 4 and 6-9 appear to have no effect on the level of FGFR3 induced phosphorylation (upper panel) and were given a score of 0, while compounds 2 and 3 almost completely reduce the amount of the phosphorylated receptor and were ranked as 4. An intermediate effect was observed with compound 5 (ranked as 2). The effect of the compounds on downstream signaling was assessed by evaluating the level of activated Jun kinase (pJNK, lower panel).

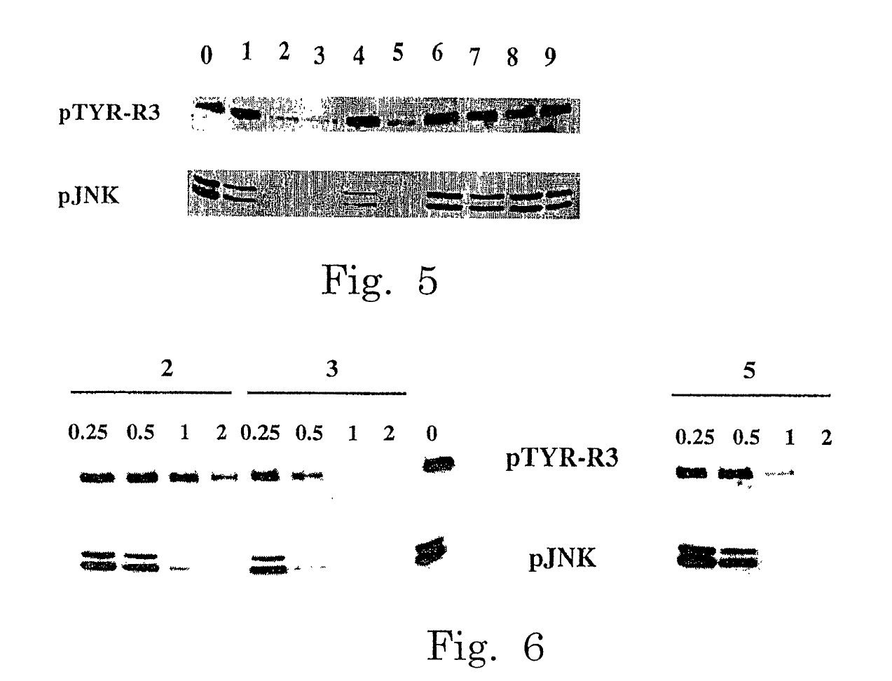

The compounds 2, 3 and 5 that gave significant level of inhibition of receptor phosphorylation and activation of JNK, were further analyzed for dose dependent inhibition, using the same assays. An example of dose response inhibition of FGFR3 phosphorylation and signal transduction is shown in FIG. 6. Analysis of the inhibitory compounds 2, 3 and 5 was performed by the use of 0.25-2 μM of these compounds for each sample as indicated. The cells were exposed to the compounds 20 minutes before stimulation with FGF9. Cells were then harvested and subjected to immunoprecipitation with anti-FGFR3 antibodies and probed with anti phosphotyrosine antibodies (upper panel). Total cell lysates were also analyzed for inhibition of JNK activation (lower panel). As shown, the degree of inhibition by the compounds differs between the tested compounds. While compound 2 inhibited 50% of receptor tyrosine phosphorylation at a concentration of 1.5 μM, compound 3 was much more potent and inhibited receptor phosphorylation at 0.25 μM.

### Example 4

Generation of RCJ Cell Lines Expressing FGFR1 and FGFR2

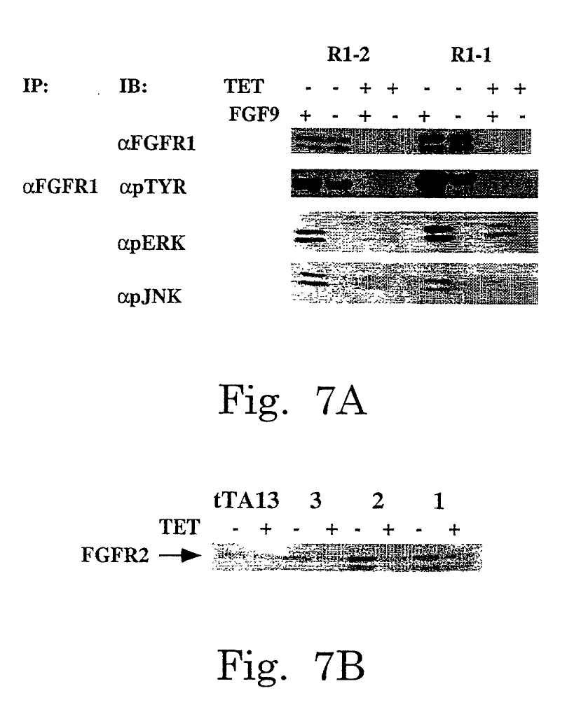

In order to determine the specificity of the inhibitory compounds for FGFRs, we have also generated stable cell lines expressing FGFR1 and FGFR2. FIG. 7 shows measurement of signal transduction proteins in such cell lines.

Expression of wild type FGFR1 in RCJ clones was analyzed by probing the Western blots with anti-FGFR1 specific polyclonal antibodies in the presence and in the absence of tetracycline. Two of the selected FGFR1 clones, R1-1 and R1-2, are shown (FIG. 7A). Basal and FGF9 induced tyrosine phosphorylation of FGFR1 was determined by immunoprecipitation of the receptor with antibodies to FGFR1 and immunoblotting with antibodies to phosphotyrosine. Analysis of the signaling pathways regulated by FGFR1 was performed by removal of tetracycline and then stimulating the cells with FGF9 and probing with antibodies directed to the activated forms of ERK and JNK, as indicated.

The level of FGFR1 expression, as determined by Western blotting with anti-FGFR1 antibodies, was shown to be tightly regulated by tetracycline (FIG. 7A). FGFR1 is tyrosine phosphorylated upon stimulation with FGF9, leading to activation of ERK and JNK.

RCJ clones expressing wild type FGFR2 were generated in a similar way. Expression level of FGFR2 in the absence of tetracycline is shown in FIG. 7B. FGFR2 was shown to be tightly regulated by tetracycline in these clones.

These cells may be used for the screening of FGFR1 and FGFR2 inhibitors. They may also be used for in vivo screening in animal models.

### Example 5

Effect of FGFR3 Expression on Tumorigenic Potential in an Animal Model Using L8 Cells

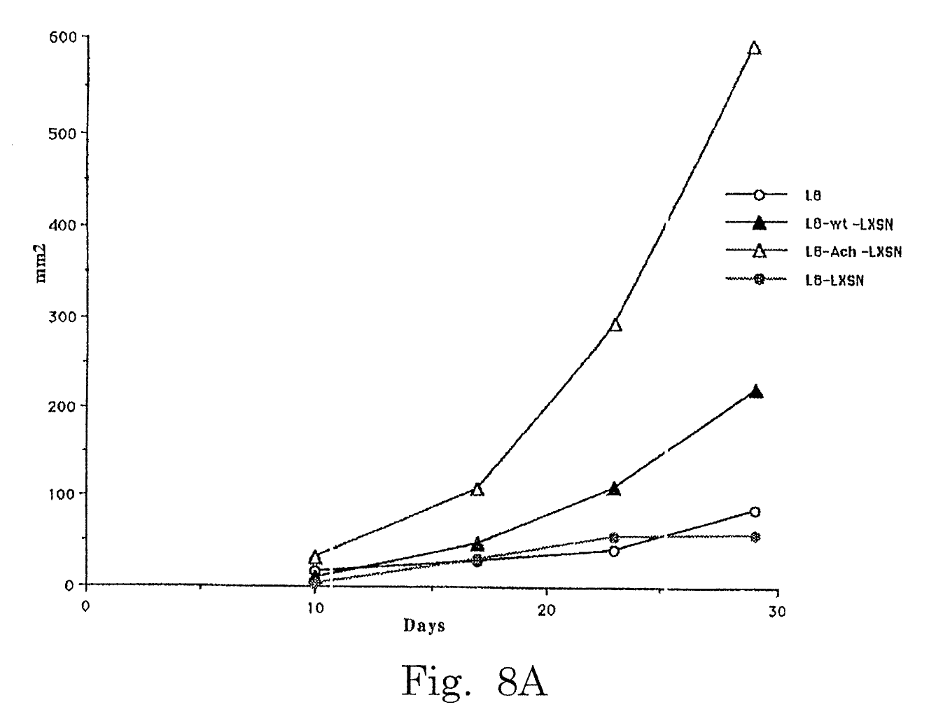

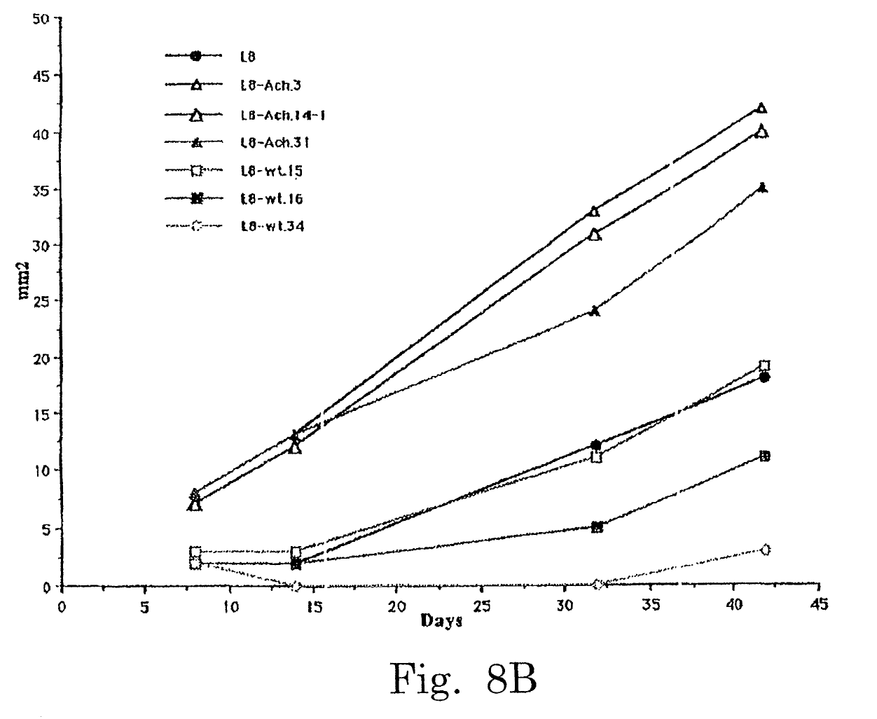

L8 cells expressing recombinant wild type or mutant G380R FGFR3 were established by infecting L8 cell pools with retrovirus pLSXN. Retroviruses, expressing either wild type or mutant FGFR3, were produced by transient transfection, essentially as described before (Ausubel, F. M., Brent, R., Kingston, R. E., Moore, D. D., Seidman, G. G., Smith, J. A., Struhl, K. (1999) Current Protocols in Molecular Biology (John Wiley & Sons, Inc.). In short, by using the CaCl₂ method, 293 cells were co-transfected with pLXSN expressing either wt or mutant FGFR3, and Psi minus helper vector. 24-hr post transfection, supernatant was removed and replaced by fresh medium. 48-hr later retroviral supernatant was collected and filtered through a 0.45-um filter. The retroviral sups were either used immediately or stored in aliquots at −80° C. About 2×10⁶ L8 cell pools infected with pLSXN derived virus expressing the FGFR3, in 250 μl PBS, were injected subcutaneously into each mouse. Tumor formation and size were followed for five weeks after the injection. FIG. 8 shows the results as a time course for tumor development showing the mean for each cell type, i.e., either stable lines (FIG. 8 B) or for LSXN-infected cell pools (FIG. 8A).

It is evident from the data that expression of FGFR3 receptor in the L8 cells dramatically changes their tumorigenic potential. Expression by retroviral vector (FIG. 8A) enhances the tumorigenic potential of the L8 cells, independent of whether wild type or mutant receptor was used. Expression of FGFR3 in stable cell lines by transfection with pcDNA3 containing the wt FGFR3 showed a clear tendency to suppression of tumor formation (FIG. 8B, circles, squares). On the other hand, expression of FGFR3 in stable cell lines by transfection with pcDNA3 containing the G380R mutated FGFR3, clearly enhanced tumor formation in the mice.

This assay is therefore a useful in vivo model for screening for inhibitors of the FGFR3 pathway. In addition, FGFR1 or FGFR2 expressing cell lines or retrovirally infected cell pools may be prepared according to the invention and used in the same screening assay. It is expected that FGFR1 and FGFR2 have the potential to change the tumorigenic potential of mesenchymal cells, and this may be used in an in vivo screening assay according to the invention, for the identification of FGFR1 or FGFR2 antagonists.

Thus, when adding a compound that is an antagonist by injection into the animal or addition to the animal's food or drinking water, the antagonist will inhibit the FGFR3 pathway and therefore suppress tumor formation in this L8 model, where expression of FGF receptor enhances tumor formation. Conversely, as shown in Example 6 thereafter for the RCJ cells, where FGFR expression inhibits tumor formation, administering a compound which is an antagonist of this FGFR will enhance tumor formation in this assay.

### Example 6

Tumor Suppressor Activity of FGFR3 in an Animal Model Using RCJ Cells

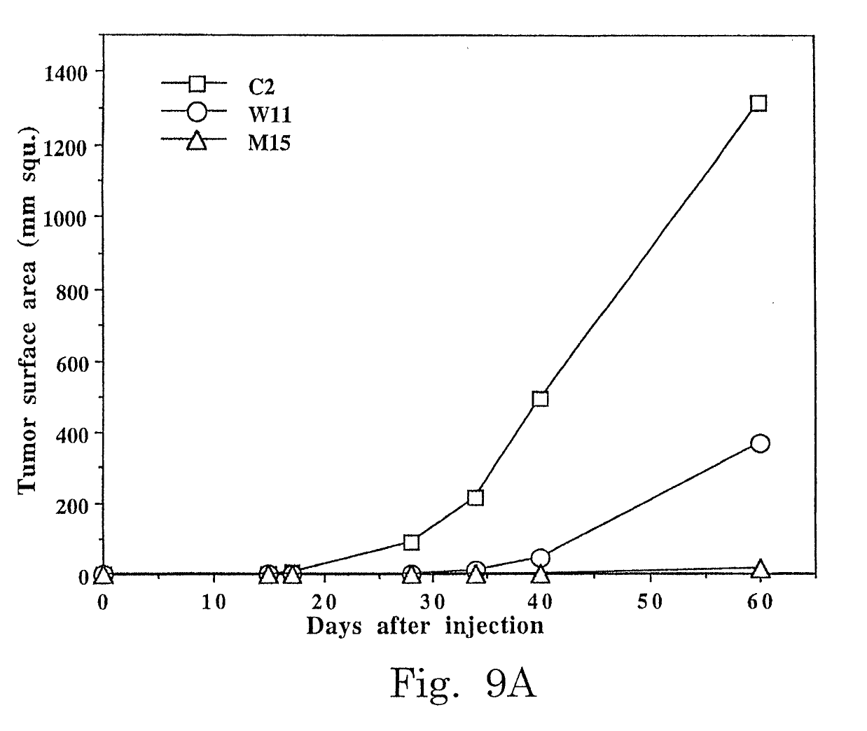

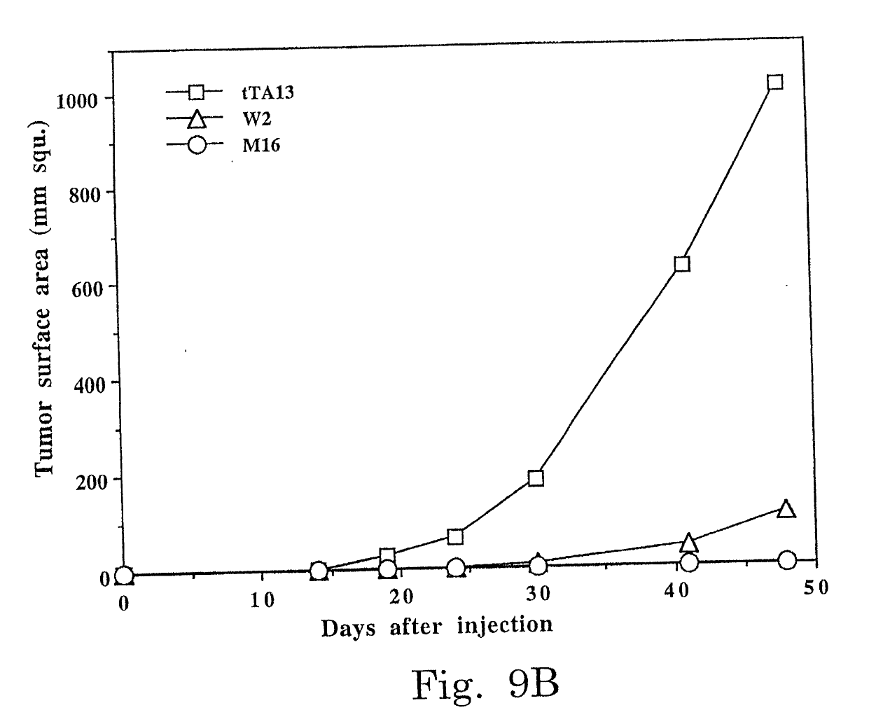

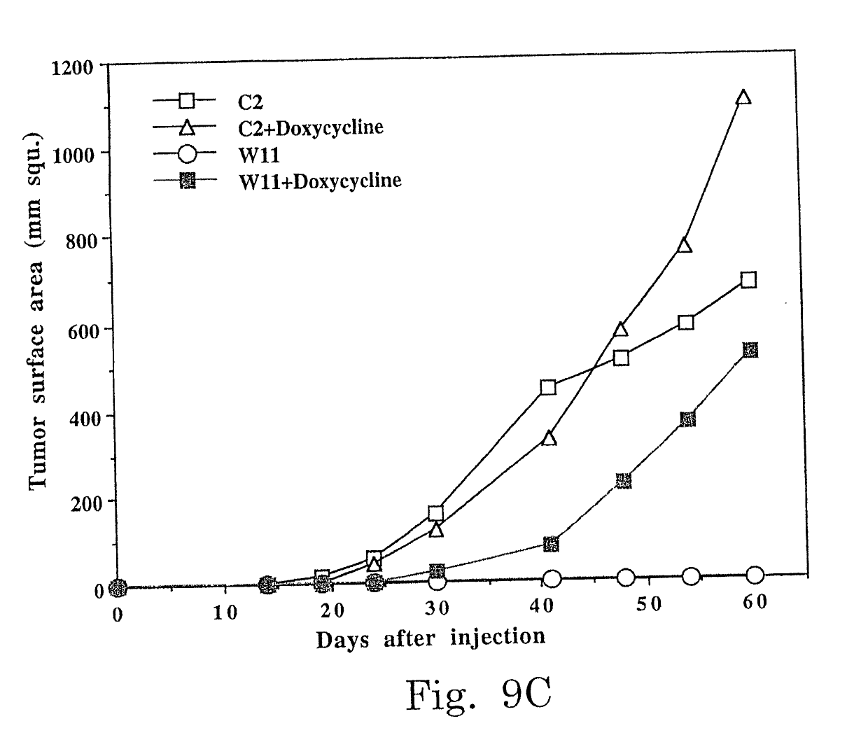

Nude mice were injected subcutaneously with equivalent numbers of parental RCJ cells (FIGS. 9 A-C, C2, squares) or wild type or mutant FGFR3 expressing RCJ cell lines (W11, circles in FIG. 9A, M15, triangles in FIG. 9A, W2, triangles in FIG. 9B, M16, circles in FIG. 9B) and tumor surface areas were evaluated for up to 60 days after injection (ordinates). FIG. 9C shows induction of tumor formation by addition of doxycycline to the drinking water of the mice. Triangles, C2 (control) clone plus doxycycline, circles, W11 clone, filled squares, W11 clone plus doxycycline Doxycycline was used in animals because the former is a potent high affinity derivative of tetracycline.

As shown in FIGS. 9A and 9B, the parental RCJ cells lead to the formation of large solid tumors, while overexpression of either wild type or mutant FGFR3 significantly suppressed tumor formation and progression. The inhibition of tumor formation of RCJ cells was highly correlated with the expression levels of FGFR3. In order to determine whether inhibition of tumor formation reflects the expression level of FGFR3, we have performed an assay where we tried to suppress the expression of FGFR3 in vivo by the addition of doxycycline to the drinking water of injected mice. The results are shown in FIG. 9C. While the presence of doxycycline in the drinking water of the mice injected with the parental line had no significant effect on tumor formation, the presence of doxycycline had a very dramatic effect on tumor formation (FIG. 9C). Wild type FGFR3 expressing cells, which without doxycycline did not form significant tumors, gave rise to relatively large tumors in mice treated with 25 μg/ml of doxycycline, probably by suppressing the levels of the expressed receptor. From the results it is conceivable to suggest that FGFR3 negatively regulates cell proliferation and tumor formation of RCJ cells.

This model is ideal for the analyses of modulators of FGFR1, FGFR2 and FGFR3 in vivo. This model is unique in that it represents a double specificity stringent assay for the identification of FGFR1, FGFR2 and FGFR3 specific inhibitors as only such compounds will enhance tumor formation and progression in animals bearing RCJ cells expressing recombinant FGFR1, FGFR2 or FGFR3. This is an artificial model for the screening. However, these inhibitory compounds will not themselves be tumorigenic.

### Example 7

FGFR3 Inhibitor Screening Assay Using Chondrocyte Differentiation as a Marker

FGFR1-, FGFR2- and FGFR3-transfected chondrocytic cell lines according to the invention undergo differentiation in the presence of any FGF ligand. This FGFR effect may be used, in accordance with the teaching of the invention, to screen for FGFR1, FGFR2 and FGFR3 antagonists.

In one example, the RCJ-W11 clone, expressing wild type FGFR3, may be used for screening compounds that affect chondrocyte differentiation mediated by FGFR3. The FGFR3 expressing RCJ cells aggregate and form cartilage nodules in the presence of FGF. A screening assay is thus established wherein RCJ-W11 cells are cultured in the presence of FGF. The compounds to be evaluated are added to the culture and nodule formation is evaluated by light microscopy after about three weeks. Cell aggregation may also be measured by flow cytometry, turbidimetry, and similar methods. Compounds that inhibit cell aggregation are FGFR3 antagonist candidates.

It may be seen from FIG. 10 that cells of the W11 clone clearly form cartilage nodules when receptor FGFR3 expression is induced in the absence of tetracycline (right hand panel), as compared to the dispersed appearance of these cultures in the presence of tetracycline (i.e. repressed receptor expression; left hand panel). The upper panels of FIG. 10 show the cells viewed under phase contrast microscopy, while in the lower panels, cells were viewed following fixation and Safranin-O staining.

While specific embodiments of the invention have been described for the purpose of illustration, it will be understood that the invention may be carried out in practice by skilled persons with many modifications, variations and adaptations, without departing from its spirit or scope of the claims.

What is claimed is:

1. An in vitro screening assay for antagonists of FGFR-mediated malignant cell transformation comprising the steps:
(i) providing a stable cell line genetically engineered to express a recombinant fibroblast growth factor receptor (FGFR) selected from FGFR1, FGFR2 and FGFR3, wherein the malignant potential of said cell line is modulated by said FGFR;
(ii) subjecting said cell line of (i) to treatment with the corresponding FGF ligand and a candidate antagonist; and
(iii) measuring an FGFR downstream signaling event, wherein an antagonist is identified by suppressing said FGFR downstream signaling event.

2. The screening assay of claim 1 wherein the FGFR downstream signaling event is selected from:
(a) FGFR tyrosine phosphorylation;
(b) activation of one or more intracellular proteins involved in signal transduction pathways of receptor tyrosine kinases selected from STAT1, JNK, PLCγ, ERK, STAT5, PI3K, PKC, FRS2 and/or GRB2; and/or
(c) a cell differentiation-related effect.

3. The screening assay of claim 1 wherein the FGFR is FGFR3, the cells are treated with FGF9 and a candidate antagonist, and the downstream signaling event is activation of JNK, and the antagonist inhibits said JNK activation.

4. The screening assay of claim 1 wherein the FGFR downstream signaling event is a cell differentiation-related effect selected from cell aggregation, formation of nodules and/or formation of cartilage, said effect being detected by light microscopy, turbidimetry, or flow cytometry.

5. The screening assay of claim 1 wherein the FGFR downstream signaling event is a cell differentiation-related effect being a change in the expression at RNA or protein levels of a marker of cell differentiation selected from bone sialoprotein, matrilin-3, type X collagen, 4-1BB, ILA, type II collagen and/or MGP.

6. The screening assay of claim 1, wherein the cell line is genetically engineered by transfection or infection with an expression vector containing a DNA encoding the wild type FGFR1, FGFR2 or FGFR3 or a constitutively active mutant thereof.

7. The screening assay of claim 6, wherein said cells are transfected or infected with an expression vector containing a DNA encoding the wild type FGFR1, FGFR2 or FGFR3.

8. The screening assay of claim 6, wherein said cells are transfected or infected with an expression vector containing a DNA encoding a constitutively active mutant FGFR1, FGFR2 or FGFR3.

9. The screening assay of claim 8, wherein said cells are transfected or infected with an expression vector containing a DNA encoding the constitutively active mutant FGFR3 comprising the G380R substitution.

10. The screening assay of claim 6, wherein said wild type or mutant FGFR is expressed in said genetically engineered cells under the control of a non-regulatable promoter.

11. The screening assay of claim 6, wherein said wild type or mutant FGFR is expressed in said genetically engineered cells under the control of a regulatable promoter.

12. The screening assay of claim 11, wherein the regulatable promoter is selected from a tetracycline-responsive and a tetracycline-repressible promoter.

13. The screening assay of claim 1, wherein said cell line is derived from muscle tissue.

14. The screening assay of claim 13, wherein the cell line is a myoblast cell line.

15. The screening assay of claim 14, wherein the cell line is derived from the L8 myoblast cell line.

16. The screening assay of claim 15, wherein the L8 myoblast cell line is genetically engineered by transfection or infection with an expression vector containing a DNA encoding the wild type FGFR3 or the constitutively active mutant FGFR3 comprising the G380R substitution.

17. The screening assay of claim 1, wherein said cell line is derived from a chondrocyte cell line.

18. The screening assay of claim 17, wherein the chondrocyte cell line is derived from an RCJ cell.

19. The screening assay of claim 18, wherein the chondrocyte RCJ cell line is genetically engineered by transfection or infection with an expression vector containing a DNA encoding the wild type FGFR1, FGFR2, or FGFR3 or a constitutively active mutant thereof.

20. The screening assay of claim 18, wherein the chondrocyte RCJ cell line is genetically engineered by transfection with a plasmid or infection with a retroviral vector containing a DNA encoding the wild type FGFR3 or the constitutively active mutant FGFR3 comprising the G380R substitution.

21. The screening assay of claim 19, wherein the genetically engineered chondrocyte RCJ cell line expressing FGFR1, FGFR2 or FGFR3 is a cell line deposited at the CNCM under Accession Nos. I-2122, I-2123, I-2124, or I-2125, and progenies thereof.

22. An in vivo screening assay for antagonists of FGFR-mediated malignant cell transformation and tumor formation and progression, comprising the steps:
(i) providing a stable cell line genetically engineered to express a recombinant wild type or constitutively active mutant fibroblast growth factor receptor (FGFR) selected from FGFR1, FGFR2 and FGFR3, wherein the malignant potential of said cell line is modulated by said FGFR;
(ii) implanting or injecting said cells of (i) expressing said recombinant FGFR into a non-human animal;
(iii) administering a candidate antagonist to said animal, either concomitantly with said cells of step (ii) or thereafter; and
(iv) evaluating the formation of tumors in said animal, wherein an antagonist is identified as a suppressor of FGFR-induced tumor formation and progression or as an enhancer of FGFR-suppressed tumor formation and progression.

23. The in vivo screening assay of claim 22, wherein the animal is a mammal.

24. The in vivo screening assay of claim 23, wherein the mammal is a mouse.

25. The in vivo screening assay of claim 23, wherein the immune system of said mammal is deficient in one or more aspects.

26. The in vivo screening assay of claim 25, wherein the animal is a SCID or nude mouse.

27. The in vivo screening assay of claim 26, wherein genetically engineered rat myoblast L8 cells expressing a recombinant constitutively active mutant FGFR selected from FGFR1, FGFR2 or FGFR3, are implanted or injected into a nude mouse, and the FGFR-induced tumor formation and progression of said FGFR-expressing L8 cells is inhibited by administration of an inhibitor of FGFR to the mouse thus causing a decrease in tumor formation and progression in the mouse.

28. The in vivo screening assay of claim 27, wherein said genetically engineered rat myoblast L8 cells express the recombinant constitutively active mutant G380R FGFR3.

29. The in vivo screening assay of claim 28, wherein said genetically engineered myoblast L8 cells are the cells herein designated L8-hAchR3, deposited at the CNCM under Accession No. I-2382.

30. The in vivo screening assay of claim 26, wherein genetically engineered rat chondrocyte RCJ cells expressing a recombinant wild type or constitutively active mutant FGFR1, FGFR2, or FGFR3, are implanted or injected into a nude mouse, and the FGFR-suppressing tumor formation of said FGFR-expressing RCJ cells is inhibited by administration of an inhibitor of FGFR thus causing an increase in tumor formation and progression.

31. The in vivo screening assay of claim 30, wherein said genetically engineered rat chondrocyte RCJ cells express the recombinant constitutively active G380R mutant FGFR3.

32. The in vivo screening assay of claim 29, wherein said genetically engineered rat chondrocyte RCJ cells are cells expressing the G380R mutant FGFR3, wild type FGFR1, wild type FGFR2, and wild type FGFR3, herein designated RCJ-13 M14, RCJ-13 R1-1, RCJ-13 R2-2, and RCJ-13 W11, respectively, deposited at the CNCM under Accession Nos. I-2122, I-2123, I-2124, and I-2125, respectively.

33. A stable cell line whose malignant phenotype is modulated by a FGFR selected from FGFR1, FGFR2 and FGFR3, said cell line being selected from genetically engineered rat myoblast L8 cells and rat chondrocyte RCJ cells expressing a recombinant wild type or constitutively active mutant FGFR1, FGFR2 and FGFR3 under a regulatable or a non-regulatable promoter, and progenies thereof.

34. The stable cell line according to claim 33, wherein said cells are genetically engineered rat myoblast L8 cells expressing the wild type FGFR3 and the G380R mutant FGFR3, herein designated L8-hWTR3-34 and L8-hAchR3, respectively, and deposited at the CNCM under Accession Nos. I-2381 and I-2382, respectively, and progenies thereof.

35. The stable cell line according to claim 33, wherein said cells are genetically engineered rat chondrocyte RCJ cells expressing the G380R mutant FGFR3, wild type FGFR1, wild type FGFR2, and wild type FGFR3, herein designated RCJ-13 M14, RCJ-13 R1-1, RCJ-13 R2-2, and RCJ-13 W11, respectively, deposited at the CNCM under Accession Nos. I-2122, I-2123, I-2124, and I-2125, respectively, and progenies thereof.
